(19) United States
(12) Patent Application Publication
(10) Pub. No.: US 2021/0307949 A1
(43) Pub. Date: Oct. 7, 2021

(54) Traction-Apparatus to Decompress Body's Joints

(71) Applicant: Sastry K. Ganti, Chicago, IL (US)

(72) Inventor: Sastry K. Ganti, Chicago, IL (US)

(21) Appl. No.: 16/837,254

(22) Filed: Apr. 1, 2020

(51) Int. Cl. A61F 5/042; A61H 1/02; A63B 21/065; A61G 13/12; A61G 13/00; A63B 21/00

(52) U.S. Cl. CPC A61F 5/042; A61H 1/0229; A63B 21/065; A61G 13/126; A61H 2203/0456; A61G 13/121; A63B 21/151; A63B 21/4013; A63B 21/4017; A61G 13/009

(57) ABSTRACT

A traction apparatus develops a pull force through some or all the compressed joints of the ankles, the knees, the hips and the vertebrae of an individual,
after the individual attaches to the ankles, or the waist, or the shoulders, a harness connected to suspended weights,
and then positions self to rest supine over an inclinable, full length frictionless surface of full-width idler rollers,
and inserts self's head's occipital portion into a crescent shaped hollow head restrainer which arrests the body's pull by the weights.

### CROSS-REFERENCE TO RELATED APPLICATION

This application claims the benefit of provisional patent application, serial number U.S. 62/860,066, filed Jun. 11, 2019, confirmation number 1244, EFS ID 36269162

### FEDERALLY SPONSORED RESEARCH

Not applicable

### SEQUENCE LISTING PROGRAM

Not Applicable

### BACKGROUND OF—THE FIELD OF INVENTION AND PRIOR ART

(In all that follows, ‘Person’ refers to the individual desirous of Traction on self)

### Field of Invention

The Field of Invention comprises a Traction-Apparatus, which with minor variations, can apply a pull (i.e. joint stretch, traction force) to one to three regions of the body:
(1) all of body's compressed joints, namely those at ankles, knees, hips and all the vertebrae (All-Joints), or selectively to
(2) all the vertebral joints only (Vertebral-Joints), or selectively to
(3) the joints in the cervical vertebrae only (neck region) (Cervical-Joints), for the Person who may accomplish the traction application by self or with assistance.

The pull force is obtained by (the body) being attached to suspended weights. The attaching points are the ankles (All-Joints), or the waist (Vertebral-Joints) or the shoulders (Cervical-Joints) using specific harnesses.

The Person, all the while, lies supine on a (inclinable) frictionless bed having the Person's head held steady to the pull.

### Prior Art

Prior art U.S. Pat. No. 8,157,757 falls short in several areas as discovered after constructing prototypes and testing. (figure numbers given in this section refer to prior art). At the foot end, i.e. the traction force generation end, the coupled objects to generate traction are cumbersome, not easy to implement, handle and manipulate. They are not

easy to couple or decouple to and from the limbs of the person in a simple easy way; the methods illustrated to apply traction result in pinch points and bending moments on feet, and result in heavy localized friction spots on the ankles (FIG. 1, 2, 3, 5 etc.).

The friction on tables over rollers, i.e. resting surface under the limbs and torso of the person, is excessive due to sliding surfaces and it absorbs large amount of applied force, rather than transmitting it through to the joints (FIG. 2, 5, 7 etc.). The result is excessive traction requirement at ankles, to begin with, to obtain adequate traction at the cervical vertebrae.

The restraint mechanisms at the person's head to counter the applied traction force are inadequate, interfering with the jaw and sight, and face access. The designs shown are unsafe due to several pinch points on face and head, with reaction contacting regions inefficiently located. The reactive load is not safely distributed at the reacting regions of the person's head. The illustrated arrangements cause bending of the head and discomfort for larger traction forces. Any reactive load applied at the jaws can be painful. (FIG. 2, 8 etc.).

The apparatus is expensive, cumbersome, heavy and elaborate to build (FIG. 1,2 etc.). Safety to the person may become an issue since the person cannot instantly negate the traction to zero by removing jaw restraint to lift head and terminate the application. The apparatus is not easily inclinable to diminish friction and aid traction to the person's body. The process of the person lying down on the traction bed may not be easily self-assisted in several occasions. Traction weights are not standardized. Rollers under the bed's flat surfaces create numerous pinch trap points on frame for small objects dust, etc. The apparatus is difficult to maintain and clean, especially in a consumer's home environment.

The apparatus is not economical for consumers to purchase or implement.

### Background of the Invention—Objects and Advantages

The Objects encompassing all the three variations of Traction-Apparatus are as follows:
(1) Traction-Set(s) (two, one per leg, for All-Joints, as required for Vertebral-Joints, none or as required, for Cervical-Joints)
   A Traction-Set typically comprises:
   A Weights-Set and a Rope-Apparatus for All-Joints, A Weights-Set and a Waist-Harness for Vertebral-Joints, A Weights-Set and a Shoulder Harness for Cervical-Joints. (for cervical-Joints, an optional method needs no Traction-Set at all)
   Weights-Set comprises Weights-Container, Container-Weights, Container-Cover,
   Sling and a Hook.
   For All-Joints, Two Rope-Apparatuses, one per leg, with strategically formed series of Overhand Knots, each knot followed by an Eyelet. A Rope-Apparatus ends with a Long Eyelet, Last-Knot and a pair of Laces.
   For Vertebral-Joints, a Waist-Harness hooked to Weights-Set(s) as required.
   For Cervical-Joints, a Shoulder-Harness hooked to Weights-Set(s) as required.
(2) An Idler-Bed composed of wide width frictionless parallel Idler-Rollers, mounted in a Roller-Frame which in turn is mounted on a sturdy, (Inclinable) Bed-Frame. The Bed-Frame has Side-Rails for support, and a Sub-Frame at the Head-End.
(3) At the Head-End of Sub-Frame is an open (fall through space) crescent shaped Head-Restrainer.

The Advantages of the present invention are as follows:

The apparatus as described needs no power connections and it is efficient in form and function. The Apparatus has superior safety features, such as simple act of sitting up to terminate an application, any time as desired.

The inclinability of the bed facilitates bringing any undesirable frictional resistances to zero or below very easy. With extra inclination of bed, and ease of finetuning the inclination, the Person's body weight may be used to develop traction for cervical joints.

Traction-Sets are modularized with all their parts, low in profile for easy, efficient body hook ups, and pre-prepping, to conserve cycle time.

With the flat-topped Container-Cover used as a footrest, and the Idler-Rollers used as a stool for sitting, the need for any side equipment (such as a stool) is eliminated.

In case of Cervical-Joints, use of extra inclination to generate traction by using the Person's body weight, eliminates any harness as well as Traction-Sets.

The full body width Idler-Rollers at all points of body support bring comfort to the Person lying on rollers.

The Head-Restrainer's curved shape to distribute the reactive load and the cushion padding, maximizes contact area of the Person's skull (occipital region), and that makes heavier (beneficial) traction loads to be applied for longer periods.

An individual of any height can simply choose the right Rope-Apparatus and the most beneficially positioned eyelet for hooking the weights quickly.

All harness hook ups direct the load into the body efficiently, symmetrically through the longitudinal axis of the body, directing the forces on foot along the centers of the lower limbs, on the torso and neck through the vertebra, with no bending, twisting and pinching.

The freewheeling Idler-Rollers reduce loss of traction to friction to near zero, and light inclination of the Head-End up takes friction to below zero.

The open crescent shaped (fall through space type) Head-Restrainer allows for comfortable backward tilt of the neck, which facilitates better resistance at the occipital bone to traction loads. With light inclination set to the apparatus, any residual friction against applied traction can be eliminated and thus the pull force amounts at any joint is predictably equal to the applied load.

### SUMMARY

The Traction-Apparatus is a simple, efficient, safe apparatus in form and function needing no power, having the ability to decompress selectively cervical, vertebral or all compressed joints of one's body to any safe level, for as long as desired, at any time

desired and at any repetition of application desired. It comprises three primary components:
1—Traction-Sets to provide the pull at joints, 2—inclinable Roller-Bed of idler rollers for one to lie supine on a frictionless surface, 3—contoured padded Head-Restrainer to hold the head steady with its occipital bone region resisting the pull force.

### DRAWINGS, FIGURES

FIG. 1 Traction-Apparatus, set Horizontal, All-Joints, Side Rails omitted

FIG. 2 Bed-Frame with Sub-Frame and Roller-Bed, set Inclined

FIG. 3 End View of Bed-Frame, Roller-Bed, Roller-Frame

FIG. 4 Traction-Set (Weight-Set & Rope-Apparatus), for All-Joints

FIG. 5 Two ropes together forming an Overhand Knot

FIG. 5A Symbolic simplified view of an Overhand Knot

FIG. 6 Enlarged view, Laces crossing over and under

FIG. 6A Symbolic view, Laces crossing over and under

FIG. 6B Laces crossing each other

FIG. 7 Rope-Apparatus construction details, two styles

FIG. 8 Regions of the Foot/Ankle (right foot shown), ref.
Ankle Wrap Steps A1, A2, A3, A4, A5 shown in FIGS. 9, 10, 11, 12 (lace pull at each step is the pull of the remainder of a lace after previous step)

FIG. 9 Wrap Step A1, diagonal up pull of laces to rear of foot, then laces crossed over and under at surface above heel ball at Achilles

FIG. 10 Wrap Step A2, horizontal forward pull of laces, then laces crossed over and under, over surface on medial dorsal region

FIG. 11 Two similar Wrap Steps A3, and A4, of the two laces, pulled diagonally forward and up, ‘after’ routing them under and over the diagonal laces of Wrap Step A1, near and below medial and lateral malleolus prominences

FIG. 12 Wrap Step A5 vertical pull up of laces, laces crossed over and under, shoe laces style bow tie knot over surface on posterior dorsal region
Ankle Wrap Steps B1, B2, B3, B4 shown in FIGS. 13, 14, 15, 16 (lace pull at each step is the pull of the remainder of laces after previous step)

FIG. 13 Wrap Step B1, vertical pull up of laces, crossed over and under over surface on medial dorsal region

FIG. 14 Wrap Step B2, diagonally down and back pull of laces below, crossed over and under the malleolus prominences, at surface above the heel ball at Achilles

FIG. 15 Wrap Step B3, diagonally down and forward pull of laces, crossing each other at surface under foot at medial plantar region

FIG. 16 Wrap Step, B4 vertically pull up of laces, shoes style bow tie knot over posterior dorsal region

FIG. 17 Open (fall through spaced) crescent shaped Head-Restrainer

FIG. 18 Waist Harness for Vertebral-Traction

FIG. 19 Shoulder Harness for Cervical-Traction

FIG. 20 Cervical-Traction using bed's extra inclination

FIG. 21 Alternate embodiment using Shoe-Sock ankle wrap concept for All-Joints

FIG. 22 Under arm Traction-Absorber, with partially compressed Traction-Bracket

FIG. 23 Traction-Absorber positionally adjusted based on Person's height

### DRAWINGS, REFERENCE NUMERALS

| | |
|---|---|
| Traction-set, All-Joints | 10 |
| Roller-Bed | 12 |
| Bed-Frame | 14 |
| Head-Restrainer, (hollow open space) crescent shaped bracket | 16 |
| Angle of Head-Restrainer to Vertical, nominally at 90 deg. | 17 |
| Sub-frame | 18 |
| Person in traction, lying supine | 20 |
| Incline-block | 22 |
| Shim | 24 |
| Foot-End | 26 |
| Head-End | 28 |
| First-roller | 30 |
| Last-Roller | 32 |
| Gap of no support, optional, under neck | 34 |
| Idler-Roller | 36 |
| Roller-Frame | 38 |
| Side Rail | 40 |
| Shoe-Sock | 50A |
| Shoe-Sock Eyelet | 50B |
| Shoe-Sock Lace | 50C |
| Over Under Shoe-Sock Lace at surface above heel ball at | 50D |
| Achilles | |
| Normal over under followed by shoe style knot with Shoe- | 50E |
| Sock | |
| Shoe-Sock Hook | 50F |
| Weights-Apparatus | 52 |
| Weights-Container | 54 |
| Container-Weights | 56 |
| Container-Cover | 58 |
| Sling | 60 |
| Hook | 62 |
| Rope Apparatus, draped over First-Roller | 64 |
| Typical Overhand Knot, shown symbolic | 66 |
| Typical Eyelet | 68 |
| Preferred Eyelet for Person | 69 |
| Penultimate Knot | 70 |
| Last-Long-Eyelet | 72 |
| Last-Knot | 74 |
| Rope to form Overhand Knots, Eyelets and Laces | 75 |
| Lace, the ends of ropes, for Ankle-Wrap | 76 |
| Laces crossed over and under, symbolic | 77 |
| Laces crossed over and under | 78 |
| Laces crossing each other | 79 |
| Single-Rope-Construction Rope-Apparatus | 80 |
| Folded half point, Single-Rope-Construction | 81 |
| Two-Rope-Construction of Rope-Apparatus | 82 |
| Aligned rope ends, Two-Rope-Construction | 83 |
| 1^{st} Eyelet, either-Rope -Construction | 84 |
| 1^{st} Knot, either-Rope -Construction | 88 |
| Overland knot followed by Shoe Style Butterfly Knot | 94 |
| Surface under foot, at medial plantar region | 98A |
| Surface above heel ball, at Achilles | 98B |
| Surface at Heel Ball of foot | 98C |
| Surface of medial malleolus prominence | 98D |
| Surface of lateral malleolus prominence | 98E |
| Surface on posterior dorsal region | 98F |
| Surface on medial dorsal region | 98G |
| Head-Restrainer's Sturdy Core Bar | 100 |
| Padding at Head-Restrainer Bar | 102 |
| mounting bracket of Head-Restrainer | 104 |
| Waist Harness | 106 |
| Normal Inclination, for friction thresh hold just below zero | 108 |
| Traction limited to all Vertebral-Joints | 110 |
| Shoulder Harness | 112 |
| Traction limited to approximately Cervical Region | 114 |
| Large Inclination, to create traction from body weight | 116 |
| Under arm Restrainer-Bracket | 118 |
| Traction-Absorber with under arm Restrainer-Bracket | 120 |

### DETAILED DESCRIPTION, OPERATION

Description: Note: Though the Person (20) in some figs may be shown with nothing worn on feet, in reality the Person (20) should always wear heavy (cotton, tube style) fabric socks so as to avoid any unintended pinch forces of laces, harnesses, etc. when in traction.

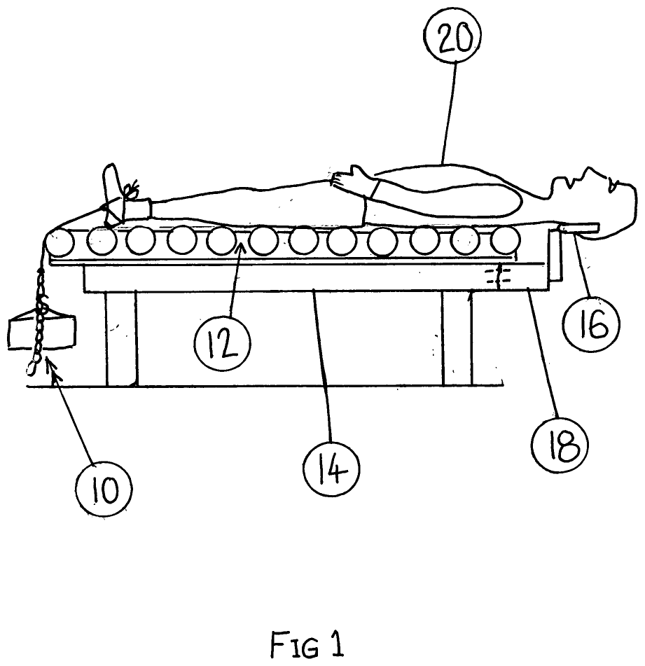

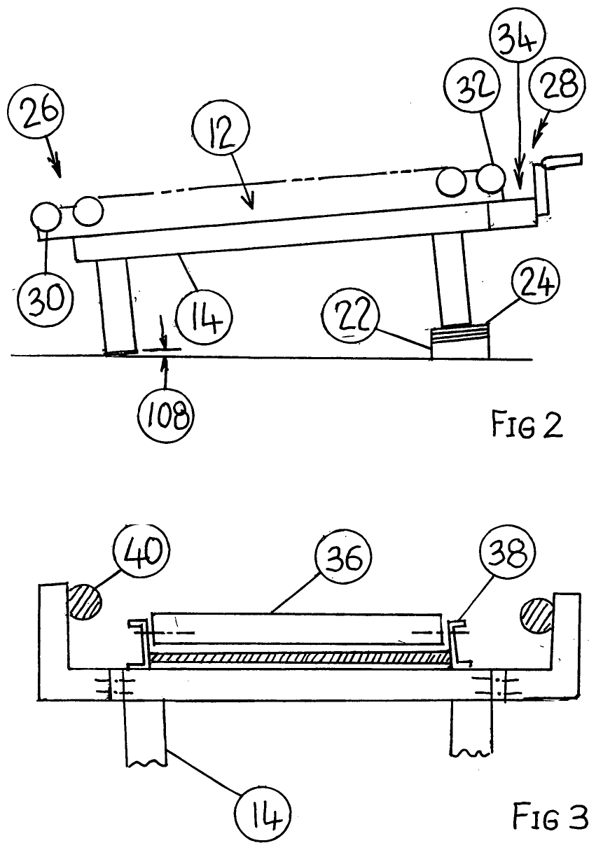

In FIG. 1 the Person (20) desirous of traction application is shown for reference, lying supine on the traction apparatus Roller-Bed (12). The sturdy Roller-Bed (12) is mounted on a sturdy and easily inclinable Bed-Frame (14). Suspended from each ankle of the Person (20) is one Traction-Sets (10), for All-Joints. Sub-Frame (18), mounted to Bed-Frame (14) at the Head-End (FIG. 2,28) carries mounted to it the crescent shaped open space Head-Restrainer (16). The setup shown is horizontal. The top of the Head-Restrainer

(16) is in level or slightly below the top of the roller-bed (12) surface.

Note: when the bed is horizontal, though each idler is virtually frictionless, the number of rollers under contact with the Person, and the softness of physicality of the Person's back, together, add a certain ‘tare’ amount of pull load which really is not part of traction. Knowing and subtracting this amount (by various methods) from applied traction allows the true effective traction force to be computed.

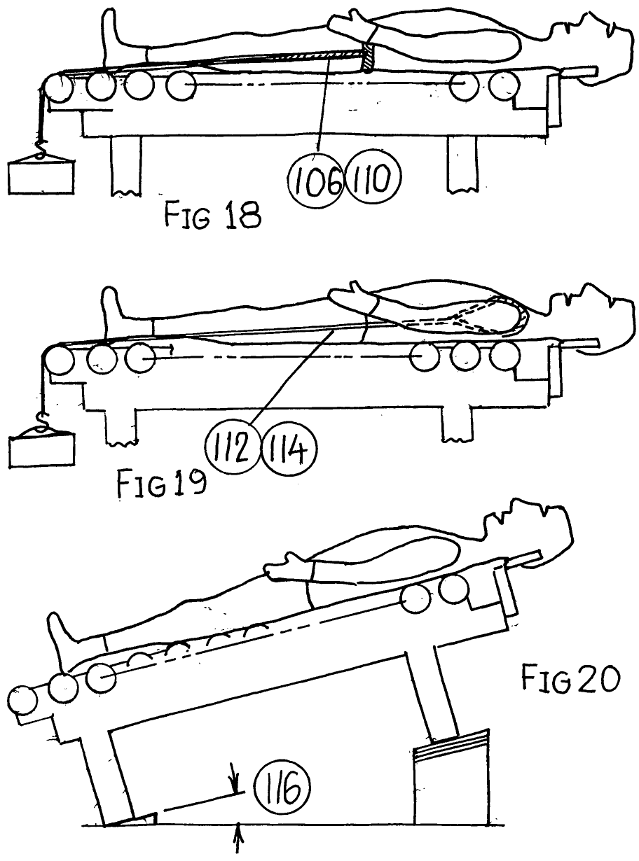

FIG. 2 shows The Bed-Frame (FIG. 1-14) shown inclined by using Incline-Blocks (22). Inclination is fine tuned to requirements of threshold of zero friction or other by using Shims (24). At this angle the Person would tend to slide down with no traction applied. The Head-End (28) will always be higher than the Foot-End (26) if inclined. The First-Roller (30) functions as a freewheeling pulley for draping the ropes of Traction-Set (FIG. 1, 10) or any other harness in case of Vertebral-Joints or Cervical-Joints Traction (FIGS. 18, 19). There may be some intentional gap (34) between the last roller (32) and the Head-Restrainer (16) designed to be right under the neck of the person.

FIG. 3 shows the easily removable, replaceable frictionless Idler-roller(s) (36) that is sprung-snapped (collapsible axles) into a unitized Roller-Frame (38). Side-rails (40) assist the Person to mount or de-mount the Roller-Bed (FIG. 1,12). As a rule, hands should be off of them for effective traction, during traction. Whenever Traction-Sets are used, inclination (108) is intended to be to small, only to the extent that a body becomes self-sliding.

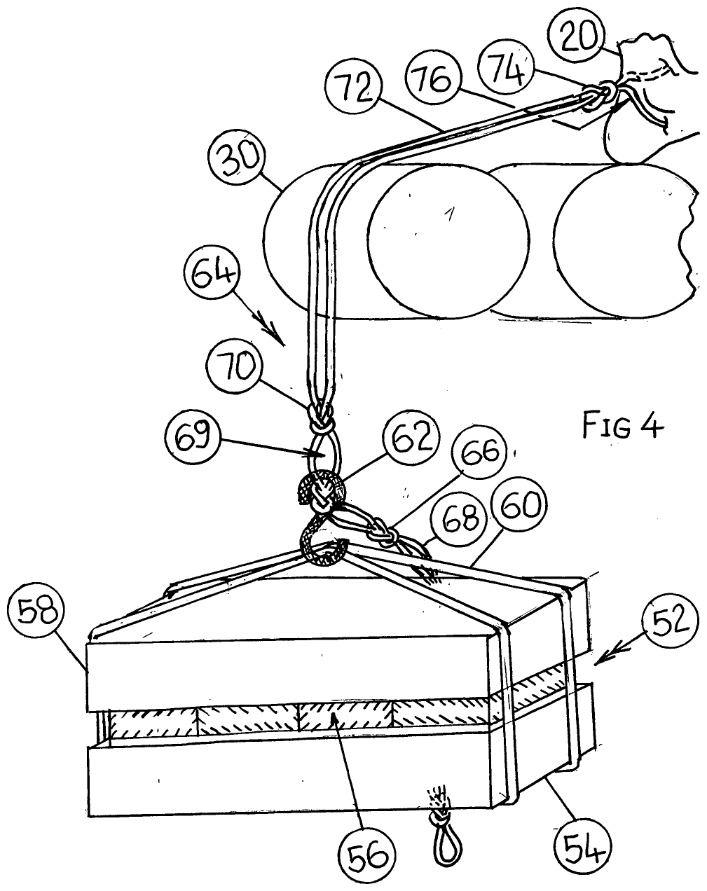

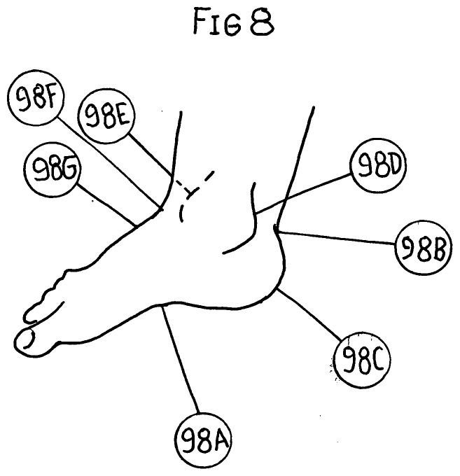

Traction-sets as required (FIGS. 1, 10 & FIG. 4) normally are placed on floor at bed's Foot-End (FIG. 2, 26), fully pre-prepared and ready to go for ankle wrapping for All-Joints. In cases Vertebral-Joints or Cervical-Joints, the Rope-Apparatuses (64 or FIG. 7 80, 82) may be used in conjunction with Waist-Harness (FIG. 18, 106, 110) or Shoulder-Harness (FIG. 19, 112, 114). The Hook connection to Rope-Apparatus Eyelet (FIG. 4, 62) may be premade or made after all connection at the Person's body are made.

FIG. 4 describes the details of a Traction-set (10), as used for All-Joints. Each Traction-Set (FIG. 1, 10) comprises a Weight-Set (52) with required Weights-Container (54), Container-Weights (56), Container-Cover (58), Container-Sling (60), and a Rope-Apparatus (64) with multiple hook up positions as Eyelets (68) Note: in lieu of inclining the apparatus to bring friction to zero, a required weight can be added to the Container-Weights bringing the friction prior to traction application to zero or less. This weight of course is discounted as a traction force.

Weight-Set (FIG. 4, 52): The Weights-Container (54) contains the needed Container-Weights (56) loosely placed, as chosen by the Person (FIG. 1, 20) or a responsible

individual. Container-Cover (58) is placed on top of Container-Weights (56); it serves also as a footrest during ankle wrapping of laces or harness connections. It can also act as an (assisted) placement location for additional weights on its top, as needed, as an afterthought. The Sling (60) securely envelops the Weights-Container (54) for suspended lift through S shaped Hook (62) anchored on top of a chosen, typical Knot (66, 62).

Rope-Apparatus (FIG. 4, 64 and FIG. 7, 80, 82): The upper end of the Hook (62) is inserted into a chosen eyelet (69) to latch on against the Knot immediately below. The Eyelet (69) is so chosen that the last Long-Eyelet (72) between Penultimate-Knot (70) and Last-Knot (74) drapes over the First-Roller (30) at the Foot-End (FIG. 2, 26) after the Person has fully positioned self into Traction (FIG. 1, 20). Laces (76), sufficiently long, are available for ankle wrapping (All-Joints).

For harness use for Vertebral or Cervical Traction, the Traction-Set with its Rope-Apparatus may be connected to suitable additional harnesses, or the harness may directly connect to the Weight-Set.

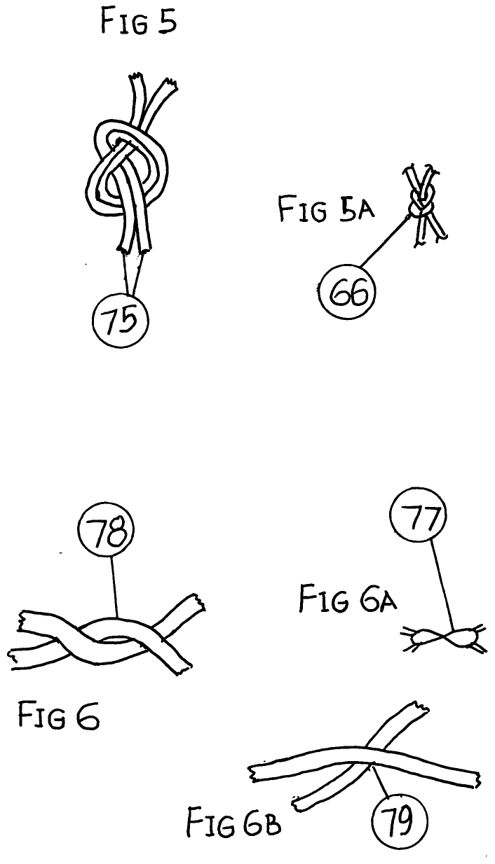

FIG. 5 shows the routing of Ropes (75) to form Overhand Knots (FIG. 5A,66). Pulling the ends of the Ropes as directed forms a ‘hard’ difficult to unravel Overhand Knot (66). FIG. 5A shows symbolic representation of a ‘hard’ Overhand Knot (66)

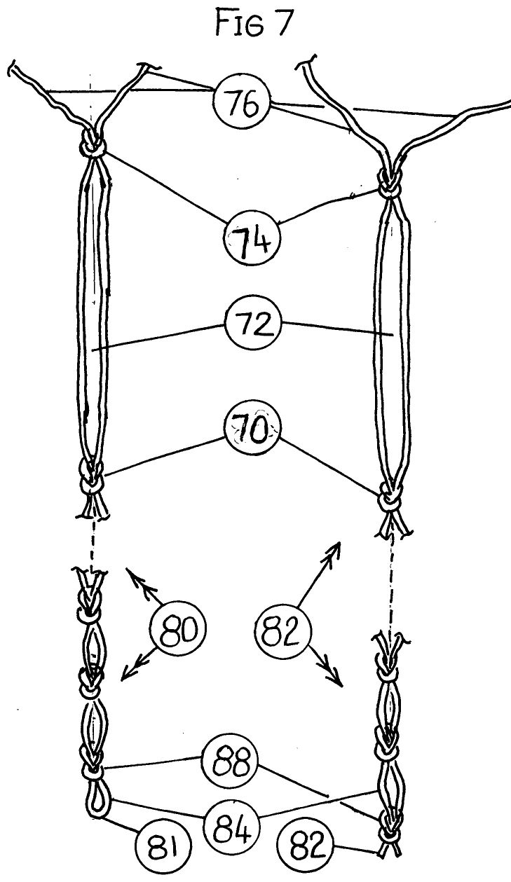

FIG. 6 shows the crossing over and under of the Laces at various ankle wraps (78). FIG. 6A (77) is a symbolic representation of (78). FIG. 6B shows Laces simply crossing over each other (79).

Note: at the ankle wrap, wherever the laces are expected to go over and under (78), a simple crossing (79) may be substituted, though the former method is more assuring of lace positioning. Similarly, where the laces are expected to cross each other (79) over and under (78) may be substituted, though the former is adequate.

FIG. 7 Shows the two preferred ways (80, 82) of constructing Rope-Apparatuses (FIG. 4, 64) with both ropes together forming Overhand Knots (66), Eyelets (68), the Last Long-Eyelet (72) between Penultimate Knot (70) and Last-Knot (74) and Laces (76).

First method uses a sufficiently long rope and folding it at half point and making its First-Eyelet (84) with its First-Knot (88). Subsequent Eyelets, Knots, Penultimate Knot (70), Last Long Eyelet (72), Last Knot (74) and the remnant rope becoming Laces (76) are as shown.

Second method uses two substantially equal and, sufficiently long ropes and forming its First Knot (88), and the First-Eyelet (84). Subsequent Eyelets, Knots, Penultimate knot, Last Long Eyelet, Last Knot and the remnant rope becoming Laces is as shown.

FIG. 8 Shows various regions of the foot and ankle which are referred to explain the sequence of ankle wrapping of laces in two methods. Both methods terminate in any form of normal Bow Tie Knot used for shoes at the surface on Dorsal region.

(98A) shows Surface under foot at medial Plantar region,

(98B) refers to Surface above heel ball at Achilles,

(98C) refers to Surface at Heel Ball of foot,

(98D) points to Medial Malleolus prominence of the Tibia,

(98E) points to Lateral Malleolus prominence of the Fibula,

(98F) is the Surface on posterior Dorsal region, and

(98G) is the Surface on medial Dorsal region.

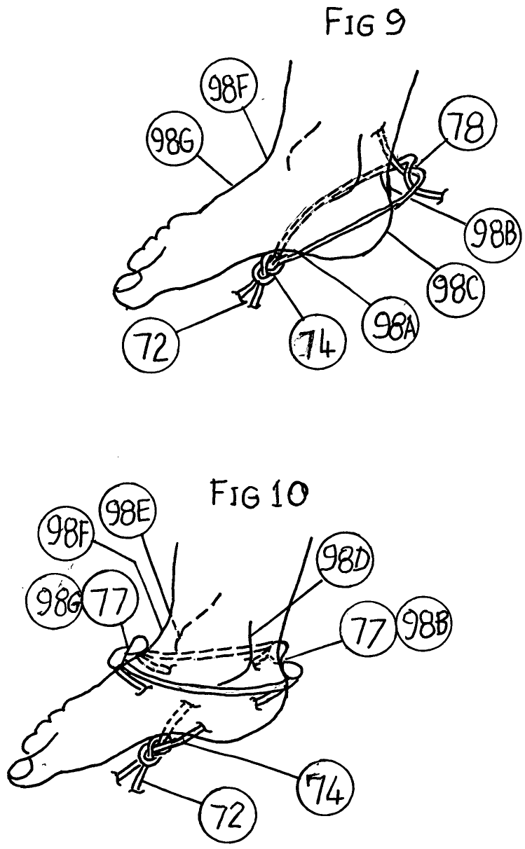

Referring to FIGS. 9, 10, 11, 12, they show ankle wraps A1, A2, A3, A4, A5 which are performed in that sequence.

The wrapping process is described in detail in operation.

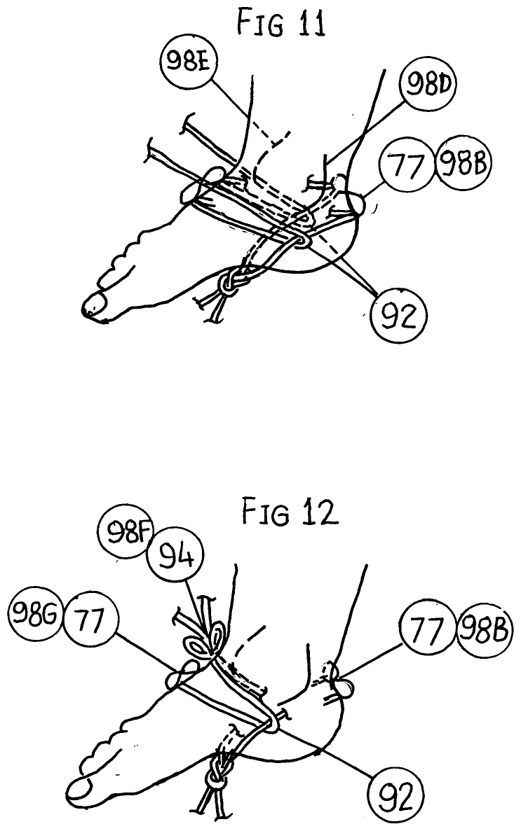

Referring to FIGS. 13, 14, 15, 16, they show ankle's alternative wraps B1, B2, B3, B4 which are performed in that sequence.

The wrapping process is described in detail in operation.

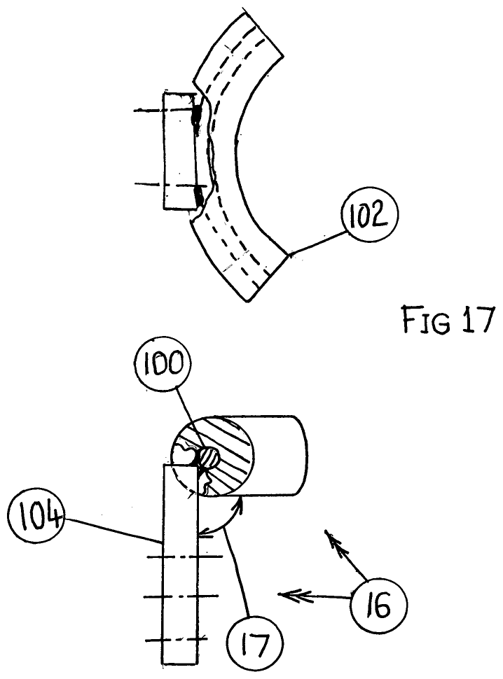

FIG. 17 shows the construction details of the Head-Restrainer (16). In the simplest form it is an arc of a circle of radius that suits the shape of the occipital bone at its lowest point region near the top of the neck for the Person, made up at its core (100) of sturdy material such as metal or strong plastic or wood or such. The strong arc shaped material is covered with softer but firm material such as rubber, foam or cloth padding (102) etc. For further comfort and hygiene, a personal piece of cloth such as a small towel can always be draped over the padding (102).

The Head-Restrainer (16) is provided with a permanently attached bracket (104) to mount it on Sub-Frame (FIG. 1, 18). After initial adjustment of its proper positioning, the Head-Restrainer (16) needs no further repositioning. The Head-Restrainer's arc profile, though shown horizontal, may be constructed at a different angle than (FIG. 17, 17) the 90 deg Shown, to suit the occipital bone's orientation.

An upgrade to the circular arc could be individualized contoured curvature part for a particular individual's occipital bone shape. In such a situation, the Subframe would be Traction-Apparatus to Decompress Body's Joints Sastry K Ganti Page 19 of 36

provided with an easily removable, securely attachable individualized head restrainer apparatus, and this is not illustrated.

FIG. 18 shows the Person lying supine on the Roller-Bed with a Waist-Harness (106) hooked to a Weight-Set, for Vertebral-Joints (110)

FIG. 19 shows the Person lying supine on a roller-bed with a shoulder-harness (112) hooked to a weights-set, for Cervical-Joints (114)

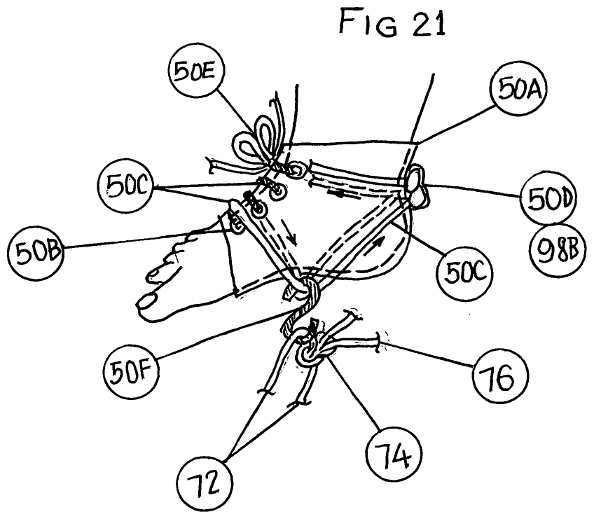

FIG. 20 points to the Person lying supine on an (extra) inclined (116) roller-bed with no attachments, basically only for Cervical-Joints. Caution is recommended with this set up. It is recommended to check the efficacy of the inclination by attaching a hard block with flat surfaces, of known weight, attached to a fish weighing scale. Place the block on the idler rollers and hold the scale steady but parallel to rollers in the sliding direction. The indicated value on the scale is the traction ‘due to the weight of the block’.

Operation: The final aim of the operations is, for the Person (20) to self-attain the position of in-traction as depicted in FIG. 1 for All-Joints, or FIG. 18 for Vertebral-Joints, or either FIG. 19 or 20 for Cervical-Joints, then stay in-traction for as long as desired, then simply sit up and slide down with the support of side rails (FIG. 3, 40) when done.

Occasionally the Person may require assistance to get to the stated situations.

The operation and use of the traction-apparatus may be described in four primary steps.

(All-Joints is described below, and Vertebral-Joints and Cervical-Joints can be followed in similar way)

1 Pre-preparation and pre-positioning of the two Traction-Sets (10),

2 Ankle wrapping of laces (76)

2 Getting into traction (20)

3 Terminating traction (simply sitting up, not illustrated)

1 Pre-Preparation involves, Refer to FIG. 4; The Weights-Containers (54) are placed at convenient spots under the First-Roller (FIGS. 1 & 2) on floor. They are filled with the desired Container-Weights (56) and covered with Container-Covers (58).

The Hook (62) may be attached to the Sling (60) at this stage, or later.

The Person (FIG. 1, 20) should wear one, or double up with two, preferably of heavy cotton, long tube, socks. Such will eliminate any uncomfortable pinching when an ankle is not wrapped correctly. Depending on the height of the Person falling into a certain height range, a particular suitable Rope-Apparatus (FIG. 7) is chosen, that which will have, in traction position, its Last-Long-Eyelet (72) draped over the First-Roller (30), so that no Knot (66, 70) will fall over any roller or the gaps between rollers. This assures maintaining frictionless condition. The preferred Eyelet (68) of the Rope-Apparatus is identified, so chosen that based on the height of the person, with the normal height increase when lying down and

the subtraction for height of head above top of head taken into account, the Weight-Set lifts off of the floor sufficiently for effective traction for All-Joints.

The Weight-Sets can be pre-hooked securely to the Rope-Apparatus at this stage, or, if more comfortable, this can be postponed to after ankle wrapping in step 2.

2 To Get into traction: Ankle wrapping is the next step.

Based on the suggestion that, the Person sit on the 1^{st} few rollers at Foot-End (26) of the Roller-Bed, set one foot at a time on one Container-Cover (58) to gain foot elevation and wrap one ankle at a time by the preferred steps A1, A2, A3, A4, A5, or B1, B2, B3, B4. These steps are explained in detail below.

The Last-Knot (74) is pulled firmly against the surface under foot at medial Plantar region (98A) as shown in all FIGS. 9 through 16, with one Lace pulled on each side of the foot (such pulling is followed before A1 or B1 steps). Then, performing (Wrap steps A1, A2, A3, A4, A5)

Step A1, pull laces diagonally up from 98A towards rear of foot, then cross laces over and under (78) at surface above heel ball at Achilles (FIG. 9, 98B), followed by,

Step A2, a horizontal forward pull of laces below bone prominences (98E, 98D), then laces crossed over and under (77) at the surface on medial Dorsal region (FIG. 10, 98G), followed by,

Steps A3, and A4, for the two laces, pulled diagonally forward and up, ‘after’ routing under and over the diagonal laces of Step A1 (FIG. 11, 92), near but below medial and lateral malleolus prominences (FIG. 11, 98D, 98E), followed by,

Step A5, a substantially vertical pull up of laces, and performing a shoe laces style Bow Tie Knot (FIG. 12, 94, 98F) at surface on posterior Dorsal region, or perform,

### (Wrap Steps B1, B2, B3, B4, an Alternate Method)

Step B1, pull laces substantially vertically up cross over and under (FIG. 13, 77, 98G) at the surface on medial Dorsal region, followed by,

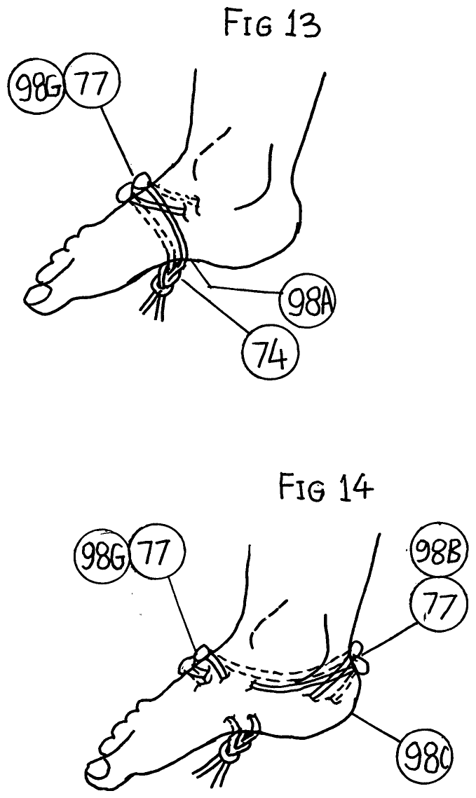

step B2 pull laces diagonally down and back crossed over and under (FIG. 14, 77, 98B) at surface above heel ball at Achilles while routing below the bone prominences, followed by,

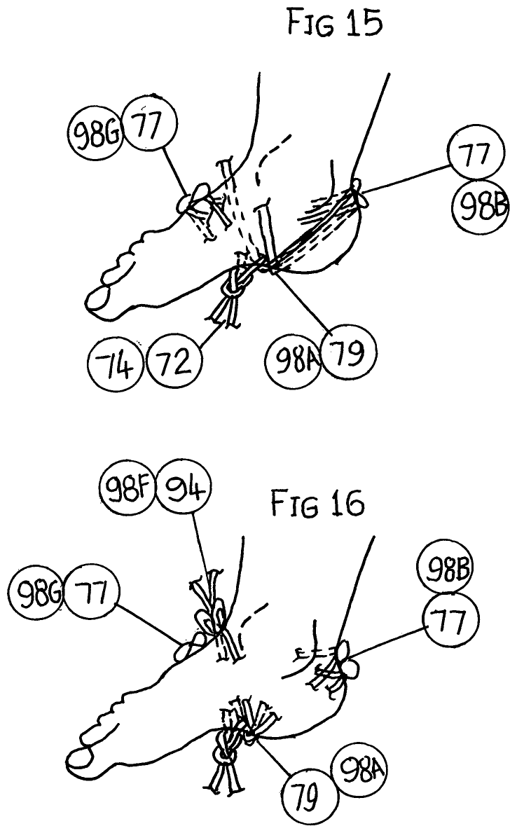

Step B3 pull laces diagonally down and forward crossing each other (FIG. 15, 79, 98A) at surface under foot at medial Plantar region, followed by,

Step B4 pull laces vertically, and crossing over and under and tie shoe style Bow Tie Knot (FIG. 16, 94, 98F).

The wraps need not be tight but lightly firm, since the traction force is naturally a wrap tightening force.

When both Traction Sets (10) are thus secured to the ankles, the Person crawls back and lowers the head gently into the Head-Restrainer's crescent shaped hollow open space (FIG. 1, 16).

The person fully ankle wrapped and crawl up on the rollers while holding guard-rails, with Weight-Set lifted off of the floor at some point, the Person leans back gently inserting self's head into the Head-Restrainer.

The person is now in traction as intended (FIG. 1) and may stay in this state to any reasonable length of period with comfort.

3 Terminating traction: the person holds guard rails, raises self's head and back to sit, and gently crawls down.

Inclining the roller bed is achieved by placing two blocks under the two legs of the Bed-Frame at Head-End (FIGS. 2, 22, and 20) (116). The angle of inclination can be finetuned to desired amount by placing Shims over the blocks (FIG. 2, 24).

The ankle wrapping sequences A or B type are routed in such a fashion around the ankle that the traction-force through Last-Long-Eyelet (74) lines up with the axis of the lower limb, thus eliminating any undesirable bending forces on the feet and legs.

### General Notes on Traction:

A joint, as referred to here is primarily the tough elastic cushioning material and the region in squeeze between the two adjacent bones, i.e. material such as disc, meniscus, cartilage etc. Any muscles, tendons, and ligaments are not primary consideration for traction, though they may benefit due to secondary effects of applied traction

Joints in the person's arms, shoulders, and ribs are not included for traction.

When an individual is standing, sitting or exercising, a joint and its related bones are squeezed, at minimum due to the body's weight above the joint, but substantially more so due to bending, body motion, exercise, etc.

Body movements of exercise may benefit muscles, tendons, ligaments, etc. but generally always traumatize the cushioning-materials with undue amounts of squeeze, friction, impediment to supply of lubricating bodily fluid, nutrition and blood flow in and removal of waste material out. The cushioning-materials generally cannot be exercised into good health.

When a body is resting as in sitting or sleeping position, though the squeeze is substantially eliminated, many joints yet being in bent condition, localized squeeze at some joints will still exist, albeit diminished substantially.

A joint and associated bones are substantially ‘fully’ devoid of the squeeze ‘only’ when they are subjected to traction, i.e. full stretching at the joint with creation of, however minute, gaps for fluid to flow in.

Applying a small amount of traction may relieve pain and discomfort at the joint, but larger (safe) amounts applied judiciously, routinely, and for reasonable duration for each traction-application, may create the required clearance (gap between bones and cushioning-material). This will facilitate easier flow of synovia, nutritional fluids and blood, thus nursing the joint into health and rebuilding. The accumulated thicknesses of the cushioning-materials and the lengths of associated bones primarily give the unique height to an individual. Both wear down and deform with age. Both may get triggered to better values with habitual judicious traction-application, depending the individual's conditions such as age, etc. Routine traction-application may correct, prevent, postpone or arrest the onset of posture deformities and certain joint deceases attributed to aging and other.

First Alternate Embodiment (FIG. 21), for ankle wrapping with laces (50C) is,

using a Shoe-Sock (50A) explained as a cross between a cutup soft shoe and a cutup (of heavy fabric, leather, vinyl, etc.) sock, with its own Shoe-Sock Eyelets (50B) and Shoe-Sock Lace (50C) and a Shoe-Sock Hook (50F).

The lacing of the Shoe-Sock lace through eyelets (50B) is shown to be reverse of a normal shoe lacing, i.e. the lacing starts at the posterior dorsal region and proceeds down to medial dorsal region, where the two substantially long laces exit to wrap over the Shoe-Sock around the ankle. Their wrapping sequence around the ankle is as shown (FIG. 21) the wrapping culminates with a normal bow tie knot (50E).

The Shoe-Sock (50A) needs to be custom fit, just as a regular shoe only a bit tighter to the human's feet. It is permanently laced (50C) through Shoe-Sock Eyelets (50B) where by default the laces are loose for wearing, and tightening before hooking (through both laces at plantar region, 98A,50F) the Shoe-Sock and the last eyelet (72) at the last Overhand Knot (74). The Rope-Apparatus's (64) Laces (76) are left unused or cut off.

In lieu of a creating a Shoe-Sock from scratch, one may create one by obtaining a smaller fit size sneakers, cut the bottoms off, reverse the lacing if need be while using only the needed eyelets, and wear it over comfortable thick cotton socks and follow the knee wrapping sequence described above.

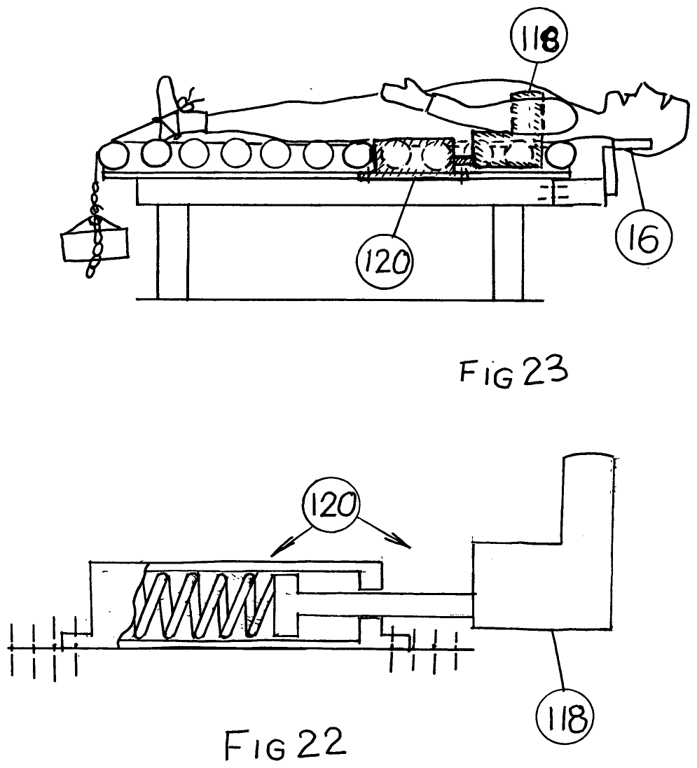

Second Alternate Embodiment (FIG. 22, 23) is, so as to accommodate a substantially large traction load that the Person desires, but the Person's occipital bone cannot handle the reaction, in addition to the crescent shaped Head-Restrainer, to install two Traction-Absorbers (120) whose vertical Traction-Brackets (118) interfere with the Person's underarms. The Traction-Brackets push sufficiently against internal resistance of the Traction-Absorbers (120). The Traction-Absorber mounting position is adjustable on the Traction-Apparatus as required based on the Person's height. Its stroke is always partial without it ever ‘bottoming’. Such a set up assures that the Head-Restrainer also remains active to resist traction.

As an example of a setup, say if the Person is desirous of a total traction of 50 pounds for All-Joints, but the Person's occipital region can withstand only 28 pounds of resistance, the Traction-Absorbers can be set to take up 11 pounds of reaction under each arm. Each joint downstream of the cervical vertebrae will take 50 pounds of traction

but each of the cervical vertebrae are under only 28 pounds of traction. This could be agreeable because the cervical vertebrae do happen to be the smallest joints among all the compressed joints in consideration needing, say, less than the lumbar vertebrae.

I claim:

1. A partial body traction apparatus for a human to lie supine, said partial body traction apparatus comprising,
a substantially flat, substantially rigid, substantially rectangular, substantially long frictionless surface to support said human's full body length, said frictionless surface being inclinable with said frictionless surface's head side elevated, and
a plurality of substantially wide frictionless idler rollers, whose crests form said frictionless surface, said idler rollers being mounted in a rigid frame over substantially the entire length of said frictionless surface, said idler rollers being arranged parallel with each other but
perpendicular to the longitudinal axis of symmetry of said frictionless surface, and
a substantially crescent shaped bracket mounted at said head side, said bracket comprising hollow open space beyond said crescent's inner, substantially cylindrical boundary surface,
whereby
said crescent shaped bracket's boundary surface will interfere with said human's occipital bone's external lower portion, to prevent said body from moving in the direction of said head side to feet side of said frictionless surface, when said human is lying as said with said human's head inserted into said hollow open space, and further,
when said frictionless surface is inclined sufficiently beyond the threshold of zero friction between underside of said human resting on said frictionless surface, with said body lying as said with said head inserted into said hollow open space, the prevention of said body's movement down by said crescent shaped bracket will create a traction force in a plurality of said body's joints due to said body's weight's component along said frictionless surface, pulling against the said inner crescent shaped boundary.

2. A full body traction apparatus for a human to lie supine, said full body traction apparatus comprising,
a substantially flat, substantially rigid, substantially rectangular, substantially long frictionless surface to support said human's full body length, said frictionless surface being inclinable with said frictionless surface's head side elevated, and
a plurality of substantially wide frictionless idler rollers, whose crests form said frictionless surface, said idler rollers being mounted in a rigid frame over substantially the entire length of said frictionless surface, said idler rollers being arranged parallel with each other but perpendicular to the longitudinal axis of symmetry of said frictionless surface, and
a substantially crescent shaped bracket mounted at said head side, said bracket comprising hollow open space beyond said crescent's inner, substantially cylindrical boundary surface, and
a means to pull said body, along said body's axis of symmetry, in the direction from said head side to the feet side of said frictionless surface, with said means to pull attached to said body anywhere between said body's shoulders to said bodies ankles inclusive of said shoulders and said ankles, whereby
said crescent shaped bracket's boundary surface will interfere with said head's occipital bone's external lower portion, to prevent said body from moving in the direction of said head side to said feet side of said frictionless surface, when said human is lying as said with said human's head inserted into said hollow open space, and further, said pull will create a beneficial traction force in said human's compressed joints located between said pull's application location and the location of the first vertebra of said body.

3. In claim 2 said means to pull comprises,
a suspended load of weights (FIG. 4, numeral 52), said weights suspended from
a rope harness (FIG. 4, numeral 64) constructed with a single rope (FIG. 7, numeral 80),
said single rope harness's construction comprising a plurality of pairs of a substantially small eyelet (FIG. 4, numerals 68, 69) with an overhand knot (FIG. 4, numeral 66), terminating with the penultimate overhand knot (FIG. 4, numeral 70) followed by the substantially long eyelet (FIG. 4, numeral 72) followed by the final overhand knot (FIG. 4, numeral 74), and with said single rope
harness terminating with a pair of laces (FIG. 4, FIG. 7, numeral 76) whereby with
said suspended load of weights (FIG. 4, numeral 52) hooked (FIG. 4, numeral 62) into one of said small eyelets (FIG. 4, numeral 69) based on said body's height, said long eyelet (FIG. 4, numeral 72) is positioned to drape over the farthest idle roller (FIG. 4, numeral 30) from said head, after said laces are attached to said body at a desired location (FIG. 1, numeral 10), while said human lies as said with said head inserted into said hollow open crescent shaped space.

4. In claim 2 the said means to pull comprises,
a suspended load of weights (FIG. 4, numeral 52), said weights suspended from,
a rope harness constructed with two ropes (FIG. 7, numeral 82),
said two ropes harness's construction comprising a plurality of pairs of substantially small eyelet (FIG. 4, numerals 68, 69) with an overhand knot (FIG. 4, 5A numeral 66, FIG. 5 75), terminating with the penultimate overhand knot (FIG. 4, numeral 70) followed by the substantially long eyelet (FIG. 4, numeral 72) followed by the final overhand knot (FIG. 4, numeral 74), and with said two ropes' harness terminating with a pair of laces (FIG. 4, FIG. 7, numeral 76), whereby with
said suspended load of weights (FIG. 4, numeral 52) hooked (FIG. 4, numeral 62) into one of said small eyelets (FIG. 4, numeral 69) based on said body's height, said long eyelet (FIG. 4, numeral 72) is positioned to drape over the farthest idle roller (FIG. 4, numeral 30) from said head, after said laces are attached to said body at a desired location (FIG. 1, numeral 10), while said human lies as said with said head inserted into said hollow open crescent shaped space.

5. A method of wrapping, the pair of said laces (FIGS. 4 and 7, numeral 76), to one of said body's foot's ankles (FIGS. 9, 10, 11, and 12), said method comprising,
bringing said human's foot's underside together with said last knot (FIGS. 4, and 9, numeral 74) in substantial proximity to said foot's plantar surface's center (FIG. 9. numeral 98A), followed by
pulling said laces one on each side of said foot (FIG. 9), in a direction substantially diagonally up and to the rear of said foot (FIG. 9) to the close proximity to said achilles (FIG. 9, numeral 98B), near the upper region of said heel ball (FIG. 9, numeral 98C), followed by
crossing said laces over and under each other (FIG. 6, 9 numeral 78,) at the upper location of said heal ball on said achilles (FIG. 9 numerals 98C, 98B), whereby said laces naturally reverse their sides on said foot (FIG. 9), followed by
pulling the said laces substantially horizontally forward on said foot to the medial dorsal surface (FIGS. 9 and 10, numeral 98G) of said foot, followed by
crossing said laces over and under each other, thus reversing sides again (FIG. 6 numeral 78, and FIG. 10, numeral 77 at numeral 98G) in relation to said foot at said medial dorsal surface (FIG. 10, numeral 98G), followed by
pulling each one of said laces, in a substantially diagonally down and rear direction (FIGS. 10 and 11), to route each one of said laces underneath the corresponding proximate lace having said diagonal up and rear direction (FIG. 11 near numeral 92), followed by a 180 degrees reversal of direction (FIG. 11, numeral 92), followed by
each of said reversed laces pulled up and forward substantially parallel to said diagonally down and rear laces (FIG. 11, near numeral 92), to the posterior dorsal surface (FIG. 12, numeral 98F) of said foot, followed by
tying a bow tie shoe knot at said posterior dorsal surface of said foot, (FIG. 12, numerals 98F and 94).

6. An alternate method of wrapping the pair of said laces (FIGS. 4 and 7, numeral 76), to one of said body's foot's ankles (FIGS. 13, 14, 15, and 16), said method comprising,
bringing said human's foot's underside together with said last knot (FIGS. 4, and 13, numeral 74) in substantial proximity to said foot's plantar surface's center (FIG. 13. numeral 98A), followed by
pulling said laces one on each side of said foot (FIG. 13), in a direction substantially vertically up to the medial dorsal surface of said foot (FIG. 13, numeral 98G), followed by
crossing said laces over and under each other (FIG. 13, numeral 77) at the medial dorsal surface (FIG. 13, numeral 98G), whereby
said laces naturally reverse their lateral and medial sides of said foot, followed by
pulling the said laces substantially horizontally to the rear of said foot (FIG. 14), in proximity to said achilles (FIG. 14, numeral 98B), at the upper region of said heel ball (FIG. 14, numeral 98C), followed by
crossing said laces over and under each other (FIG. 6, numeral 78) above said heel ball (FIG. 14, numeral 98C), followed by
pulling each one of said laces diagonally down and forward to substantially under the middle of said plantar facia of said foot (FIG. 15, numeral 98A), further pulling said laces across over each other (FIG. 6B, numeral 79) under said plantar facia to reverse their medial and lateral sides (FIG. 15, numeral 79), followed by pulling said laces substantially straight up along the sides of said foot (FIG. 15), followed by
tying a bow tie shoe knot at the surface of posterior dorsal region (FIG. 16, numerals 98F, 94).
